(19) United States
(12) Patent Application Publication
(10) Pub. No.: US 2008/0311103 A1
(43) Pub. Date: Dec. 18, 2008

(54) Anti-Inflammatory Peptides and Methods of Use Thereof

(75) Inventors: Ofer Lider, Kfar Bilu B (IL); Liora Cahalon, Givatayim (IL); Osnat Lider, Kfar Bilu B (IL); Lihi Lider, Kfar Bilu B (IL)

Correspondence Address: HESLIN ROTHENBERG FARLEY & MESITI PC, 5 COLUMBIA CIRCLE, ALBANY, NY, 12203, US

(73) Assignee: YEDA RESEARCH AND DEVELOPMENT CO. LTD. at THE WEIZMANN INSTITUTE OF SCIENCE, Rehovot (IL)

(21) Appl. No.: 10/592,163

(22) PCT Filed: Mar. 8, 2005
(86) PCT No.: PCT/IL05/00272

§ 371 Date: Jun. 1, 2007

Related U.S. Application Data
(60) Provisional application No. 60/550,307, filed on Mar. 8, 2004.

(51) Int. Cl. A61K 38/43; C12N 9/50; A61P 29/00

(52) U.S. Cl. 424/94.63; 435/219

(57) ABSTRACT

Anti-inflammatory peptides derived from naturally occurring digests of proteins including apolipoprotein A-I, apolipoprotein A-II, fibrinogen γ chain, fibrinogen Aa, low-density lipoprotein receptor, ADAM 8, cadherin 4, and calcitonin receptor are provided, along with pharmaceutical compositions comprising same and methods of treating inflammatory diseases using same.

### FIELD OF THE INVENTION

The present invention relates to peptides having anti-inflammatory activity that are derived from the amino acid sequences of known proteins identified in exudates at the site of tissue injury or trauma, to pharmaceutical compositions comprising same and to methods of treating inflammatory conditions or diseases using same.

### BACKGROUND OF THE INVENTION

Inflammation is characterized by the transendothelial migration of leukocytes from the vascular circulatory system into the extracellular matrix (ECM). This process is affected by a variety of cytokines, chemokines, and acute phase proteins situated within the context of the ECM.

The involvement of cytokines in the induction of acute inflammatory events, and in the transition to or persistence of chronic inflammation has been extensively studied. There is considerable evidence indicating that cytokines, such as tumor necrosis factor (INF)-α and interleukin (IL)-1β, contribute to the pathogenesis of inflammatory autoimmune diseases, specifically in rheumatoid arthritis (RA), multiple sclerosis (MS), systemic lupus erythromatosus (SLE), and in atherosclerosis. Recently, the secretion of TNF-α and IL-1β by monocytes was shown to be inhibited by HDL-associated apolipoprotein (Apo) A-I.

Apo A-I is a protein synthesized in man by both the liver and the intestine as a preproprotein of 267 amino acid residues, having a molecular weight of 28 kDa. The prepro form of Apo A-I is then cleaved to a proprotein, which is secreted into the plasma. In the vascular compartment, pro-Apo A-I is converted to the mature protein (243 amino acids) by the action of a calcium-dependent protease.

Apo A-I as well as another member of the apolipoprotein family, the Apo A-II, are the most prominent apolipoproteins in high-density lipoprotein (HDL), the latter is directly involved in the removal of cholesterol from peripheral tissues, carrying it back either to the liver or to other lipoproteins.

Both clinical and experimental data suggest that Apo A-I mediates the anti-atherogenic activity of HDL, and that the rate of production of Apo A-I is a critical determinant of circulating HDL cholesterol. Persons with familial hyperalphalipoproteinemia appear to be protected from atherosclerosis, while those deficient in Apo A-I show accelerated cardiovascular disease. Mice transgenic for the human Apo A-I gene demonstrate accumulation of human Apo A-I in serum, increased circulating HDL cholesterol, and resistance to the atherogenic effects, of a high cholesterol diet. Apo A-I was also found to be an activator of lecithin cholesterol acetyl transferase, an enzyme in the pathway that removes cholesterol from peripheral blood.

Variations of Apo A-I concentration were observed in several inflammatory diseases. In RA, the levels of circulating Apo A-I and HDL in untreated patients are lower than in normal controls. In addition, Apo A-I plasma concentrations are diminished in SLE and in MS patients who do not respond to interferon (INF)-β therapy. In addition, during acute inflammation the level of HDL-associated Apo A-I was found to be decreased by at least 25%. Apo A-I is, therefore, a negative acute phase protein.

The possible role of HDL associated Apo A-I in modulating inflammatory processes has been described recently. Hyka et al., demonstrated that HDL-associated Apo A-I inhibited contact-mediated activation of monocytes by binding to stimulated T cells, thereby diminishing TNF-α and IL-1β production, at the protein and mRNA levels (Hyka, N. et al., 2001). As the contact between T cells and monocyte-macrophages triggers the production of pro-inflammatory cytokines, and as Apo A-I is a natural constituent of plasma, it was suggested that Apo A-I controls the contact mediated activation of monocytes in the blood stream.

Apo A-I was also found to play a role in thrombotic events. It was shown that matrix metalloproteinases (MMPs) cleave Apo A-I into two main fragments, designated F1 and F2. The latter (the C-terminal domain) exhibits in vitro high-affinity binding to ECM components including fibrin(ogen). The F2 fragment was shown to be preferentially distributed in unstable carotid plaques. Such distribution in atherosclerotic plaques suggested that Apo A-I may compete with matrix components as a substrate for MMPs, and thus reduces plaque rupture. Though the cleavage of Apo A-I by MMPs occurs at both the amino and carboxyl terminals of the protein, the carboxyl terminus is more susceptible to MMP degradation (Eberini, I., et al. (2002) Biochem. J. 362: 627-634). The carboxyl-terminal fragments of Apo A-I were also shown to inhibit cholesterol efflux from macrophage foam cells (Lindsted, L. et al. (1999) J. Biol. Chem. 274: 22627-22634).

In contrast to negative acute phase proteins, the level of which is reduced during inflammation, the level of positive acute phase proteins is up regulated during inflammatory processes. The most prominent positive acute phase proteins are fibrinogen, C-reactive protein (CRP), amyloid A, and haptoglobin.

Fibrinogen is a 340 kDa dimeric glycoprotein consisting of a pair of three polypeptide chains Aα, Bβ, and γ that are interconnected by 29 disulfide bonds. The amino termini of these chains are joined together in a central domain that can be isolated as a single fragment from a plasmin digestion of fibrinogen. During blood coagulation, fibrinogen participates in both the cellular phase and the fluid phase of blood clot formation. As a consequence of the thrombin-catalyzed removal of fibrinopeptides A and B from the Aα and Bβ chains of fibrinogen, fibrinogen is converted into insoluble fibrin constituting the fibrin clot. The formation of a fibrin clot is a critical process in vascular repair as it facilitates cell adherence, angiogenesis and cell migration. However, in order to allow a wound healing process to proceed, the fibrin clot must be removed from the injured site. Such a process takes place as a result of fibrin degradation by the serine protease, plasmin.

Other enzymes, such as matrix metalloproteinases, were also shown to cleave fibrinogen, and the cleavage of the protein by these enzymes occurs in all the three chains leading to a significantly impaired clotting. As fibrinogen was shown to be a substrate of matrix metalloproteinases and as elevated levels of matrix metalloproteinases are detected in various pathological situations such as in rheumatoid arthritis, atherosclerosis, and tumor metastasis, it was suggested that fibrinogen cleavage products may have functional significance in inflammation, atherosclerosis, and in angiogenesis. In addition, fibrin and fibrinogen cleavage products display vasoconstricting and chemotactic activities, and are mitogenic for several cell types.

Low-density lipoprotein (LDL) receptor related protein 5 (LRP5) is a single-span transmembrane protein essential for Wnt/b-catenin signaling, likely via acting as Wnt co receptors. Signaling by the Wnt family of secreted lipoproteins plays a central role in development and disease. A key Wnt transduction pathway is the canonical β-catenin signaling, which by regulating cytosolic β-catenin protein level controls the activation of Wnt-responsive genes. Wnt signaling has been shown to be critical for proper embryonic development as well as for growth regulation of certain adult tissues. Defects in Wnt pathways have also been associated with various human cancers. However, it is only recently that a role for Wnts in the immune system has come to be appreciated. Wnts have now been shown to play significant roles in early developmental stage of both B and T lineage cells. Current studies suggest that proliferation and/or survival of these cells is associated with activation of the “canonical” Wnt/RhoA pathways.

U.S. Pat. Nos. 5,599,790, 5,919,754 and 6,265,549 to Altieri et al., relate to therapeutic compositions containing a fibrinogen homolog capable of binding to endothelial cells in an RGD-independent manner that inhibits fibrinogen binding to endothelial cells. U.S. Pat. No. 5,599,790 claims a composition comprising a therapeutically effective amount of the peptide consisting the amino acid residues 117-133 of fibrinogen γ chain having three additional amino acid residues lysine-tyrosine-glycine at the amino terminal end of the peptide, and a pharmaceutically acceptable excipient. The peptide disclosed in U.S. Pat. No. 5,599,790 has the capability to inhibit fibrinogen binding to endothelial cells. U.S. Pat. No. 5,919,754 claims a method of inhibiting fibrinogen binding to endothelial cells comprising contacting endothelial cells with a polypeptide having an amino acid residue sequence of a length of 17 to 100 amino acid residues that includes the fibrinogen γ chain residues 117-133.

U.S. Pat. No. 5,473,051 describes a specific region in the plasmin degradation product of fibrinogen (D₃₀) that binds to Mac-I receptor D₃₀ binding site on monocytes and inhibits fibrinogen binding to Mac-1 receptor via the D₃₀ binding site. U.S. Pat. No. 5,473,051 discloses polypeptides that include the amino acid segment 195-201 of fibrinogen γ chain. This segment was identified as the Mac-1 receptor binding site in fibrinogen. As Mac-1 receptor mediates monocyte adhesion to vascular endothelium, a polypeptide containing the 195-201 segment of fibrinogen γ chain may be used for inhibiting Mac-1 receptor mediated inflammation in a patient.

The present invention is based in part on the finding that proteolytic degradation of IL-2 brings about the formation of IL-2-derived peptides that exhibit anti-inflammatory activity (WO 00/11028). This finding raised the possibility that degradation of other proteins may take place at or adjacent to inflammatory sites, where sufficient amounts of proteolytic enzymes and enzyme substrates may co-exist.

There is an unmet need for medicaments useful for treating autoimmune and inflammatory diseases.

### SUMMARY OF THE INVENTION

The present invention provides peptides having anti-inflammatory activity, pharmaceutical compositions comprising same, and methods of treating or protecting against inflammatory conditions or diseases.

It is now disclosed for the first time that wound fluids present in the wounds of diabetic patients contain peptides capable of exhibiting anti-inflammatory activity. These peptides are produced naturally in situ at the site of tissue injury or trauma. The isolated peptides are derived from the amino acid sequence of naturally occurring proteins, e.g., apolipoprotein (Apo) A-I, Apo A-II, fibrinogen γ chain, fibrinogen Aα, low-density lipoprotein receptor related protein 5 (LRP5), a disintegrin and metalloprotease protein (ADAM) 8, cadherin 4, and calcitonin receptor.

The peptides exhibit anti-inflammatory activity in in vitro assay systems as exemplified by inhibition of active delayed type hypersensitivity (DTH) response, inhibition of adoptive DTH response, inhibition of T cell adhesion to fibronectin, inhibition of T cell migration toward stromal cell-derived factor, and inhibition of tumor necrosis factor (TNF)-α and interferon (INF)-γ secretion from activated T cells and monocytes.

It is further disclosed that the peptides are highly useful as anti-inflammatory agents in animal models of inflammatory diseases. The peptides are advantageous not only in treating inflammatory diseases but also in protecting against these diseases. The protective effect of the peptides increases as a function of the number of treatments given to a subject prior to the development of the inflammatory disease.

It is also disclosed that the anti-inflammatory activity of the peptides of the present invention is achieved whether the peptides are administered locally or systematically including subcutaneously or intraperitoneally.

The peptides of the present invention are highly stable against a wide variety of proteases. The stability of the peptides as manifested by their presence at the site of tissue injury or trauma is confirmed in vitro. For example, the peptide of SEQ ID NO:5, is stable even after 24 hours of exposure to serum proteases, i.e., more than 90% of the peptide are still present as an intact peptide at this time point.

According to one aspect the present invention provides a peptide having anti-inflammatory activity comprising a proteolytic fragment of naturally occurring protein, the peptide present in wound fluids at a site of tissue injury or trauma.

According to some embodiments, the peptide comprises an amino acid sequence as set forth in any one of SEQ ID NO:1 to SEQ ID NO:10, an analog, derivative, fragment, conjugate, or a salt thereof.

According to additional embodiments, the peptide comprises the sequence of amino acid residues 827 to 833 of LDL receptor related protein 5 (LRP5) as set forth in SEQ ID NO:1, an analog, derivative, fragment, conjugate, or a salt thereof.

According to further embodiments, the peptide comprises the sequence of amino acid residues 254 to 267 of apolipoprotein (Apo) A-I as set forth in SEQ ID NO:2, an analog, derivative, fragment, conjugate, or a salt thereof.

According to other embodiments, the peptide comprises the sequence of amino acid residues 96 to 106 of apolipoprotein (Apo) A-I as set forth in SEQ ID NO:3, an analog, derivative, fragment, conjugate, or a salt thereof.

According to some embodiments, the peptide comprises the sequence of amino acid residues 42 to 52 of apolipoprotein (Apo) A-II as set forth in SEQ ID NO:4, an analog, derivative, fragment, conjugate, or a salt thereof.

According to additional embodiments, the peptide comprises the sequence of amino acid residues 95 to 105 of fibrinogen γ as set forth in SEQ ID NO:5, an analog, derivative, fragment, conjugate, or a salt thereof.

According to further embodiments, the peptide comprises the sequence of amino acid residues 260 to 269 of fibrinogen Aα as set forth in SEQ ID NO:6, an analog, derivative, fragment, conjugate, or a salt thereof.

According to additional embodiments, the peptide comprises the sequence of amino acid residues 250 to 257 of fibrinogen Aα as set forth in SEQ ID NO:7, an analog, derivative, fragment, conjugate, or a salt thereof.

According to further embodiments, the peptide comprises the sequence of amino acid residues 320 to 328 of a disintegrin and metalloprotease protein (ADAM) 8 as set forth in SEQ ID NO:8, an analog, derivative, fragment, conjugate, or a salt thereof.

According to additional embodiments, the peptide comprises the sequence of amino acid residues 302 to 308 of cadherin 4 as set forth in SEQ ID NO:9, an analog, derivative, fragment, conjugate, or a salt thereof.

According to further embodiments, the peptide comprises the sequence of amino acid residues 470 to 478 of calcitonin receptor as set forth in SEQ ID NO:10, an analog, derivative, fragment, conjugate, or a salt thereof.

According to another aspect, the present invention provides a pharmaceutical composition comprising as an active ingredient a peptide having anti-inflammatory activity comprising a proteolytic fragment of naturally occurring protein, the peptide present in wound fluids at a site of tissue injury or trauma, and a pharmaceutically acceptable carrier.

According to some embodiments, the peptide having anti-inflammatory activity in the pharmaceutical composition comprises an amino acid sequence as set forth in any one of SEQ ID NO:1 to SEQ ID NO:10, an analog, derivative, fragment, conjugate, or a salt thereof and a pharmaceutically acceptable carrier or diluent.

According to additional embodiments, the pharmaceutical composition of the invention is formulated in a form selected from the group consisting of pellets, tablets, capsules, solutions, suspensions, emulsions, powders, gels, creams, suppositories, and depots.

According to a further aspect, the present invention provides a method for treating an inflammatory disease or a condition associated with inflammatory damage comprising administering to a subject in need thereof a therapeutically effective amount of a pharmaceutical composition according to the present invention, and a pharmaceutically acceptable carrier.

According to a further aspect, the present invention provides a method for protecting against an inflammatory disease or a condition associated with inflammatory damage comprising administering to the subject a therapeutically effective amount of a pharmaceutical composition according to the present invention and a pharmaceutically acceptable carrier.

According to some embodiments, the route of administering the pharmaceutical compositions of the present invention is selected from the group consisting of parenteral, oral, rectal, vaginal, topical, pulmonary, intranasal, buccal, intradermal, and ophthalmic administration. According to other embodiments, parenteral administration is selected from intravenous infusion, intraarterial infusion, intravenous injection, subcutaneous injection, intraperitoneal injection, intraarterial injection, intramuscular injection, intraventricular injection, and intralesional injection.

According to one embodiment, the subject is a mammal. According to another embodiment, the subject is a human. According to another embodiment the subject is a non-human mammal. According to another embodiment the subject is a non-mammalian vertebrate.

By virtue of their anti-inflammatory activity, the pharmaceutical compositions of the present invention can be used for treating or protecting against conditions associated with inflammatory damage. Conditions associated with inflammatory damage include, but are not limited to, skin conditions including burns induced by chemical agents, thermal stimuli, or irradiation; wounds including decubitus, ulcers, internal and external wounds, abscesses, and various bleedings; tissue damage including neuronal, neurological, skin, hepatic, nephrologic, urologic, cardiac, pulmonary, gastrointestinal, upper airways, visual, audiologic, spleen, bone, and muscle damage; tissue transplants, graft rejection; sepsis; malignant and non-malignant tumors.

Inflammatory diseases that can be treated with the pharmaceutical compositions of the invention include autoimmune and chronic degenerative diseases. Autoimmune and chronic degenerative diseases include, but are not limited to, psoriasis, rheumatoid arthritis, Crohn's disease, glomerular nephritis, autoimmune diseases such as autoimmune thyroiditis, atherosclerosis, lupus erythematosis, muscle dystrophy, multiple sclerosis, Parkinson's disease, amyotrophic lateral sclerosis, and Alzheimer's disease.

These and other embodiments of the present invention will be better understood in relation to the figures, description, examples, and claims that follow.

### BRIEF DESCRIPTION OF THE FIGURES

FIG. 1 shows the inhibitory effect of peptides Nos. 1 to 5 on delayed type hypersensitivity response in mice sensitized with oxazolone.

FIG. 2 shows the inhibitory effect of peptides Nos. 6 to 10 on delayed type hypersensitivity response in mice sensitized with oxazolone.

FIG. 3 shows the inhibitory effect of peptide No. 3 and peptide No. 5 on adoptive transfer delayed type hypersensitivity in mice.

FIG. 4 shows the inhibitory effect of peptides Nos. 1 to 5 on phorbol 12-myristate 13-acetate (PMA)-induced T cell adhesion to immobilized fibronectin.

FIG. 5 shows the effect of peptides Nos. 1 to 5 on T cell migration toward stromal cell derived factor (SDF)-1α.

FIG. 6 shows the inhibitory effect of peptides Nos. 1 to 5 on TNFα secretion by THP-1 monocytes and T cells.

FIGS. 7A-B show the inhibitory effect of peptides Nos. 2 to 10 on TNFα or INFγ secretion in anti-CD3 antibody activated T cells. FIG. 7A shows the effect of peptides Nos. 2 to 10 on TNFα secretion in anti-CD3 antibody activated T cells. FIG. 7B shows the effect of peptides Nos. 2 to 10 on INFγ secretion in anti-CD3 antibody activated T cells.

FIG. 8 shows the effect of peptide Nos. 2, 3, 5 and 6 on nuclear translocation of NF-κB in T cells. The amount of NF-κB translocated to the nucleus was determined by SDS-polyacrylamide gel electrophoresis followed by immunoblotting using anti-NF-κB. The nuclear protein Lamin B was used as a control protein for quantifying the protein amounts.

FIG. 9 shows the effect of peptide No. 3 and peptide No. 5 on inflammation scores as evaluated according to Wallace criteria in experimental model of inflammatory bowel disease (IBD) in mice.

FIG. 10A-B show the effect of peptides No. 2, 3, 5, 6, 8, and 10 on serum concentrations of alanine aminotransferase and aspartate aminotransferase in an experimental model of acute hepatitis. FIG. 10A shows the effect of the peptides after two injections, both were administered to mice before concanavalin (Con) A injection, which leads to hepatic injury. FIG. 10B shows the effect of the peptides after three injections, all were administered to the mice before concanavalin (Con) A injection.

### DETAILED DESCRIPTION OF THE INVENTION

The present invention relates to novel anti-inflammatory peptides derived from naturally occurring proteins. According to the principles of the present invention, the peptides were isolated and identified from wound fluids. The peptides are derived from naturally occurring proteins: Apo A-I, Apo A-II, fibrinogen γ chain, fibrinogen Aα, LDL receptor related protein 5 (LRP5), ADAM 8, cadherin 4, and calcitonin receptor. The peptides exert anti-inflammatory activity that is demonstrated in various in vitro and in vivo assays. The present invention further relates to pharmaceutical compositions comprising said peptides and to methods for treating or protecting against inflammatory diseases or conditions in subjects in need thereof.

Without being bound to any mechanism of action, the peptides disclosed herein may be involved in a feedback mechanism, by which the inflammatory process is arrested upon degradation of naturally occurring proteins at a site of injury, resulting in the production of the anti-inflammatory peptides of the invention. These peptides arrest the inflammatory process and consequently enable wound healing.

According to one aspect, the present invention provides a peptide having anti-inflammatory activity comprising a proteolytic fragment of naturally occurring protein, the peptide present in wound fluids at a site of tissue injury or trauma.

According to some embodiments, the peptide comprises an amino acid sequence as set forth in any one of SEQ ID NOs:1 to 10, an analog, a derivative, a fragment, a conjugate, or a salt thereof. The peptides of the invention are derived from the following naturally occurring proteins: LDL receptor related protein 5 (RLP5), Apo A-I, Apo A-II, fibrinogen γ chain, fibrinogen Aα, ADAM 8, cadherin 4, and calcitonin receptor. The amino acid sequences of the peptides of the invention are as follows:

| | | | |
|---|---|---|---|
| Peptide Number | Sequence | Derived from | Residue No. |

| | | | |
|---|---|---|---|
| 1 (SEQ ID NO:1) | MIESSMN | LRP5 | 827-833 |
| 2 (SEQ ID NO:2) | LSALEEYTKKLNTQ | Apo A-I | 254-267 |
| 3 (SEQ ID NO:3) | WDNLEKETEGL | Apo A-I | 96-106 |
| 4 (SEQ ID NO:4) | TDYGKDLMEKV | Apo A-II | 42-52 |
| 5 (SEQ ID NO:5) | NPDESSKPNMI | Fibrinogen γ | 95-105 |
| 6 (SEQ ID NO:6) | ELERPGGNEI | Fibrinogen Aα | 260-269 |
| 7 (SEQ ID NO:7) | ALTDMPQM | Fibrinogen Aα | 250-257 |
| 8 (SEQ ID NO:8) | DHSKNPVGV | ADAM 8 | 320-328 |
| 9 (SEQ ID NO:9) | NDADDST | Cadherin 4 | 302-308 |
| 10 (SEQ ID NO:10) | NQGEESAEI | Calcitonin receptor | 470-478 |

The term “peptide” is used throughout the specification to designate a linear series of amino acid residues connected one to the other by peptide bonds. The peptide according to the principles of the present invention is other than the intact protein and known fragments thereto. The amino acid residues are represented throughout the specification and claims by one-letter codes according to IUPAC conventions.

The term “amino acid” or “amino acid residue” is understood to include the 20 naturally occurring amino acids; those amino acids often modified post-translationally in vivo, including, for example, hydroxyproline, phosphoserine, and phosphothreonine.

The peptides of this invention are not limited in size. However, the invention particularly contemplates peptides having fewer than about 50 amino acid residues in total. It also contemplates proteins in which the core motif sequence, namely the amino acid sequences of the peptides of the present invention, is artificially implanted within a sequence of a polypeptide, such as peptides manufactured by recombinant DNA technology or chemical synthesis.

The peptides of the present invention can be isolated from wound fluids by any protein purification method known in the art (see, for example, Example 1 herein below). Alternatively, the proteins listed herein above can be subjected to one or more proteolytic enzymes to yield a mixture of peptides, which can further be purified by any protein purification method known in the art to obtain the isolated peptides. Alternatively or additionally, the proteins listed herein above can be cleaved by chemical agents such as, for example, CNBr to yield a mixture of peptides that can be further purified to obtain isolated peptides.

The peptides of the present invention can also be synthesized using methods well known in the art including chemical synthesis and recombinant DNA technology. Synthesis may be performed by solid phase peptide synthesis described by Merrifield (see J. Am. Chem. Soc., 85:2149, 1964).

In general, peptide synthesis methods comprise the sequential addition of one or more amino acids or suitably protected amino acids to a growing peptide chain. Normally, either the amino or the carboxyl group of the first amino acid is protected by a suitable protecting group. The protected or derivatized amino acid can then either be attached to an inert solid support or utilized in solution by adding the next amino acid in the sequence having the complimentary (amino or carboxyl) group suitably protected, under conditions suitable for forming the amide linkage. The protecting group is then removed from this newly added amino acid residue and the next amino acid (suitably protected) is then added, and so forth; traditionally this process is accompanied by wash steps as well. After all of the desired amino acids have been linked in the proper sequence, any remaining protecting groups (and any solid support) are removed sequentially or concurrently, to afford the final peptide. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide, and so forth.

A preferred method of preparing the peptide compounds of the present invention involves solid-phase peptide synthesis, utilizing a solid support. Large-scale peptide synthesis is described, for example, by Andersson Biopolymers 2000, 55(3), 227-50. Examples of solid phase peptide synthesis methods include the BOC method, which utilized tert-butyloxycarbonyl as the α-amino protecting group, and the FMOC method, which utilizes 9-fluorenylmethyloxycarbonyl to protect the α-amino of the amino acid residues, both methods are well-known by those of skill in the art.

Alternatively, the peptides of the present invention can be synthesized using standard solution methods (see, for example, Bodanszky, M., Principles of Peptide Synthesis, Springer-Verlag, 1984).

The peptides according to the principles of the invention need not be identical to the amino acid sequences set forth in SEQ ID NO:1 to 10 so long as each peptide is able to function as an anti-inflammatory peptide. Thus, the present invention encompasses analogs, derivatives, conjugates, and salts comprising the amino acid sequences of the peptides of the invention or fragments thereof so long as the analogs, derivatives, salts, conjugates, and fragments are capable of inhibiting inflammatory processes.

The term “analog” includes any peptide comprising altered sequence by amino acid substitutions, additions, deletions, or chemical modifications of the peptides listed herein above and which displays anti-inflammatory activity. By using “amino acid substitutions”, it is meant that functionally equivalent amino acid residues are substituted for residues within the sequence resulting in a silent change. For example, one or more amino acid residues within the sequence can be substituted by another amino acid of a similar polarity, which acts as a functional equivalent, resulting in a silent alteration. Substitutes for an amino acid within the sequence may be selected from other members of the class to which the amino acid belongs. For example, the non-polar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. Such substitutions are known as conservative substitutions. Additionally, a non-conservative substitution may be made in an amino acid that does not contribute to the biological activity, e.g., anti-inflammatory activity, of the peptide. It will be appreciated that the present invention encompasses peptide analogs, wherein at least one amino acid is substituted by another amino acid to produce an anti-inflammatory analog of a peptide of the invention having increased stability or longer half-life as compared to the peptide listed herein above.

While the amino acid residues of the peptide sequences set forth in SEQ ID NO:1 to 10 are all in the “L” isomeric form, residues in the “D” isomeric form can substitute any L-amino acid residue so long as the peptide analog retains an anti-inflammatory activity. Production of a retro-inverso D-amino acid peptide analog where the peptide is made with the same amino acids as disclosed, but at least one amino acid, and perhaps all amino acids are D-amino acids is well known in the art. When all of the amino acids in the peptide analog are D-amino acids, and the N- and C-terminals of the peptide analog are reversed, the result is an analog having the same structural groups being at the same positions as in the L-amino acid form of the peptide. However, the peptide analog is more stable to proteolytic degradation and is therefore useful in many of the applications recited herein.

The present invention further encompasses peptide derivatives of the peptides listed herein above. The term “derivative” refers to a peptide having an amino acid sequence that comprises the amino acid sequence of the peptide of the invention, in which one or more of the amino acid residues is subjected to chemical derivatizations by a reaction of side chains or functional groups, where such derivatizations do not destroy the anti-inflammatory activity of the peptide derivative. Chemical derivatization of amino acid residues include, but are not limited to, glycosylation, oxidation, reduction, myristylation, sulfation, acylation, acetylation, ADP-ribosylation, amidation, cyclization, disulfide bond formation, hydroxylation, iodination, and methylation.

The peptide derivatives according to the principles of the present invention also include bond modifications, including but not limited to CH₂—NH, CH₂—S, CH₂—S═O, O═C—NH, CH₂—O, CH₂—CH₂, S═C—NH, CH═CH, and CF═CH and backbone modifications. Peptide bonds (—CO—NH—) within the peptide may be substituted, for example, by N-methylated bonds (—N(CH3)-CO—); ester bonds (—C(R)H—C—O—O—C(R)—N); ketomethylene bonds (—CO—CH2-); α-aza bonds (—NH—N(R)—CO—), wherein R is any alkyl group, e.g., methyl; carba bonds (—CH2-NH—); hydroxyethylene bonds (—CH(OH)—CH₂—); thioamide bonds (—CS—NH—); olefinic double bonds (—CH═CH—); and peptide derivatives (—N(R)—CH2-CO—), wherein R is the “normal” side chain, naturally presented on the carbon atom. These modifications can occur at any of the bonds along the peptide chain and even at several (2-3) at the same time.

The present invention also encompasses those peptides in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form, for example, salts, methyl and ethyl esters or other types of esters or hydrazides. Free hydroxyl groups may be derivatized to form, for example, o-acyl or o-alkyl derivatives. The imidazole nitrogen of histidine may be derivatized to form N-im-benzylhistidine.

Also included as chemical derivatives are those peptides, which contain one or more naturally occurring amino acid derivatives of the twenty standard amino acid residues. For example: 4-hydroxyproline may be substituted for proline; 5-hydroxylysine may be substituted for lysine; 3-methylhistidine may be substituted for histidine; homoserine may be substituted or serine; and ornithine may be substituted for lysine. The peptides may also contain non-natural amino acids. Examples of non-natural amino acids are norleucine, ornithine, citrulline, diaminobutyric acid, homoserine, isopropyl Lys, 3-(2′-naphtyl)-Ala, nicotinyl Lys, amino isobutyric acid, and 3-(3′-pyridyl-Ala). The peptides may also contain non-protein side chains. In addition to the above, the peptides of the present invention may also include one or more non-amino acid monomers (e.g., fatty acids, complex carbohydrates, and the like).

Peptides of the present invention also include any peptide having one or more additions of amino acid residues relative to the sequences of the peptides listed herein above, so long as the requisite anti-inflammatory activity is maintained. The amino acid residues may be added at the amino terminus and/or carboxy terminus and/or along the peptide sequence.

The present invention also encompasses peptide fragments. The term “fragment” as used herein refers to a peptide having one or more deletions of amino acid residues relative to the sequences of the peptides listed herein above, so long as the requisite anti-inflammatory activity is maintained. The amino acid residues may be deleted from the amino terminus and/or carboxy terminus and/or along the peptide sequence.

Peptide fragments may be produced by chemical synthesis, recombinant DNA technology, or by subjecting the peptides listed herein above to at least one cleavage agent. A cleavage agent may be a chemical cleavage agent, e.g., cyanogen bromide, or an enzyme, e.g., an exoproteinase or endoproteinase. Endoproteinases that can be used to cleave the peptides of the invention include trypsin, chymotrypsin, papain, V8 protease or any other enzyme known in the art to produce proteolytic fragments.

Addition of amino acid residues may be performed within the peptides and/or at either terminus of the peptides of the invention. The addition of amino acid residues at either terminus may be useful to provide a “linker” by which the peptides of this invention can be conveniently bound to a carrier. Such linkers are usually of at least one amino acid residue and can be of 40 or more residues, more often of 1 to 10 residues. Typical amino acid residues used for linking are tyrosine, cysteine, lysine, glutamic and aspartic acid, or the like.

The present invention includes conjugates of the peptides of the invention. The term “conjugate” is meant to define a peptide of the present invention coupled to or conjugated with another protein or polypeptide. Such conjugates may have advantages over the peptides themselves. Such conjugates can be made by protein synthesis, e.g., by use of a peptide synthesizer, or by ligating the appropriate nucleic acid sequences encoding the desired amino acid sequences to each other by methods known in the art, in the proper coding frame, and expressing the chimeric protein by methods commonly known in the art.

A peptide of the present invention may also be conjugated to itself or aggregated in such a way as to produce a large complex containing the peptide. Such large complexes may be advantageous because they may have new biological properties such as longer half-life in circulation or greater activity.

The anti-inflammatory activity of the peptides of the invention can be detected in in vitro and/or in in vivo assays. Thus, detecting the anti-inflammatory activity of the peptides in in vitro assays can be performed, for example, by measuring the inhibitory effect of the peptides on T cell function such as in inhibition of T cell adhesion, inhibition of T cell migration, inhibition of TNFα secretion by co-cultures of T cells and monocytes, and inhibition of IFNγ secretion by co-cultures of T cells and monocytes. The anti-inflammatory activity of the peptides can be monitored in in vivo inflammatory conditions such as, for example, in delayed type hypersensitivity (DTH), adoptive DTH, inflammatory bowel disease, and in hepatic injury. Other animal models such as, for example, experimental autoimmune encephalomyelitis (EAE) and adjuvant arthritis (AA) can also be used for assaying the anti-inflammatory activity of the peptides.

### Pharmaceutical Compositions and Administration Routes

According to another aspect, the present invention provides a pharmaceutical composition comprising a therapeutically effective amount of a peptide having anti-inflammatory activity according to the principles of the invention and a pharmaceutically acceptable carrier. It will be understood that the pharmaceutical compositions of the present invention include the peptides as listed herein above, analogs, fragments, derivatives, and conjugates thereof. Additionally, the pharmaceutical compositions of the invention can comprise one or more of the peptides listed herein above, analogs, fragments, derivatives, and conjugates thereof.

The terms “peptide having anti-inflammatory activity” and “anti-inflammatory peptide” refer to peptides that inhibit or arrest inflammatory processes and are used interchangeably throughout the specification.

As used herein, the term “pharmaceutical composition” refers to a preparation of one or more of the anti-inflammatory peptides described herein with other chemical components such as physiologically suitable carriers and excipients. The purpose of a pharmaceutical composition is to facilitate administration of an active ingredient to an organism.

The term “pharmaceutically acceptable” means approved by a regulatory agency of the Federal or a state government or listed in the U.S. Pharmacopeia or other generally recognized pharmacopeia for use in animals, and more particularly in humans.

The pharmaceutical compositions of the present invention can be formulated as pharmaceutically acceptable salts of the peptides of the present invention. The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, and tartaric acids.

The term “carrier” refers to a diluent or vehicle that does not cause significant irritation to an organism and does not abrogate the biological activity and properties of the administered compound. An adjuvant is included under these phrases. Such pharmaceutical carriers can be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. Water is a preferred carrier when the pharmaceutical composition is administered intravenously. Saline solutions and aqueous dextrose and glycerol solutions can also be employed as liquid carriers, particularly for injectable solutions.

The pharmaceutical compositions of the invention can further comprise an excipient. Herein, the term “excipient” refers to an inert substance added to a pharmaceutical composition to further facilitate administration of an active ingredient. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose are also envisioned.

Pharmaceutical compositions of the present invention can be manufactured by processes well known in the art, e.g., by means of conventional mixing, dissolving, granulating, dragee-making, levigating, emulsifying, encapsulating, entrapping, or lyophilizing processes.

Typically, pharmaceutical compositions, which contain peptides as active ingredients are prepared as injectable, either as liquid solutions or suspensions, however, solid forms, which can be suspended or solubilized prior to injection, can also be prepared. The compositions can also take the form of emulsions, tablets, pills, capsules, gels, syrups, slurries, powders, creams, depots, sustained-release formulations and the like.

Methods of introduction of a pharmaceutical composition comprising a peptide of the invention include, but are not limited to, topical, intradermal, intramuscular, intraperitoneal, intravenous, subcutaneous, intranasal, epidural, ophthalmic, rectal, and oral routes. The pharmaceutical compositions can be administered by any convenient route, for example by infusion or bolus injection, by absorption through epithelial linings (e.g., oral mucosa, rectal and intestinal mucosa, etc.), and may be administered together with other therapeutically active agents. The administration may be localized, or may be systemic. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir. Pulmonary administration can also be employed, e.g., by use of an inhaler or nebulizer, and formulation with an aerosolizing agent.

Pharmaceutical compositions for use in accordance with the present invention thus may be formulated in conventional manner using one or more physiologically acceptable carriers comprising excipients and auxiliaries, which facilitate processing of the active ingredients into preparations that can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active ingredients with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries as desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, and sodium carbomethylcellulose; and/or physiologically acceptable polymers such as polyvinylpyrrolidone (PVP). If desired, disintegrating agents, such as cross-linked polyvinyl pyrrolidone, agar, or alginic acid or a salt thereof, such as sodium alginate, may be added.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions that can be used orally include push-fit capsules made of gelatin, as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate, and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane, or carbon dioxide. In the case of a pressurized aerosol, the dosage may be determined by providing a valve to deliver a metered amount. Capsules and cartridges of, for example, gelatin for use in a dispenser may be formulated containing a powder mix of the compound and a suitable powder base, such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with, optionally, an added preservative. The compositions may be suspensions, solutions, or emulsions in oily or aqueous vehicles, and may contain formulatory agents such as suspending, stabilizing, and/or dispersing agents.

Pharmaceutical compositions for parenteral administration include aqueous solutions of the active preparation in water-soluble form. Additionally, suspensions of the active ingredients may be prepared as appropriate oily or water-based injection suspensions. Suitable lipophilic solvents or vehicles include fatty oils such as sesame oil, or synthetic fatty acid esters such as ethyl oleate, triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the active ingredients, to allow for the preparation of highly concentrated solutions.

Alternatively, the active ingredient may be in powder form for constitution with a suitable vehicle, e.g., a sterile, pyrogen-free, water-based solution, before use.

The pharmaceutical composition of the present invention may also be formulated in rectal compositions such as suppositories or retention enemas, using, for example, traditional binders and carriers such as triglycerides, microcrystalline cellulose, gum tragacanth or gelatin.

It may be desirable to administer the pharmaceutical composition of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material. Administration can also be by direct injection e.g., via a syringe, at the site of an inflammation.

For topical application, an anti-inflammatory peptide of the invention can be combined with a pharmaceutically acceptable carrier so that an effective dosage is delivered, based on the desired activity. Accordingly, an anti-inflammatory peptide of the invention can be applied to the skin for treating wounds and skin diseases such as, for example, psoriasis. The carrier may be in the form of, for example, and not by way of limitation, an ointment, cream, gel, paste, foam, aerosol, suppository, pad or gelled stick.

For directed internal topical applications, the pharmaceutical composition may be in the form of tablets or capsules, which can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate or Sterotes; or a glidant such as colloidal silicon dioxide. When the dosage unit form is a capsule, it can contain, in addition to materials of the above type, a liquid carrier such as a fatty oil. In addition, dosage unit forms can contain various other materials which modify the physical form of the dosage unit, for example, coatings of sugar, shellac, or other enteric agents.

An anti-inflammatory peptide of the invention can be delivered in a controlled release system. For example, the anti-inflammatory peptide can be administered in combination with a biodegradable, biocompatible polymeric implant, which releases the peptide over a controlled period of time at a selected site. Examples of preferred polymeric materials include polyanhydrides, polyorthoesters, polyglycolic acid, polylactic acid, polyethylene vinyl acetate, copolymers and blends thereof (See, Medical applications of controlled release, Langer and Wise (eds.), 1974, CRC Pres., Boca Raton, Fla.). A controlled release system can be placed in proximity of the therapeutic target, thus requiring only a fraction of a systemic dose.

Pharmaceutical compositions suitable for use in the context of the present invention include compositions wherein the active ingredients are contained in an amount effective to achieve the intended purpose. More specifically, a “therapeutically effective amount” means an amount of an active ingredient (e.g., an anti-inflammatory peptide) effective to prevent, alleviate, or ameliorate symptoms of an inflammatory condition or disease or prolong the survival of the subject being treated.

Determination of a therapeutically effective amount is well within the capability of those skilled in the art, especially in light of the detailed disclosure provided herein.

The amount of an anti-inflammatory peptide of the invention which will be effective in the treatment of a particular disorder or condition will depend on the nature of the disorder or condition, and can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Therapeutically effective doses may be extrapolated from dose-response curves derived from in vitro or animal model test bioassays or systems.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration, and dosage can be chosen by the individual physician in view of the patient's condition. (See, e.g., Fingl, E. et al. (1975), “The Pharmacological Basis of Therapeutics,” Ch. 1, p. 1.)

Dosage amount and administration intervals may be adjusted individually to provide sufficient plasma or local levels of the active ingredient to induce an anti-inflammatory effect (i.e., minimally effective concentration, MEC). The MEC will vary for each preparation, but can be estimated from in vitro data. Dosages necessary to achieve the MEC will depend on individual characteristics and route of administration.

Depending on the severity and responsiveness of the condition to be treated, dosing can be of a single or a plurality of administrations, with course of treatment lasting from several days to several weeks, or until cure is effected or diminution of the disease state is achieved.

### Uses of the Peptides

According to another aspect, the present invention relates to a method of treating or protecting against an inflammation by administering to a subject in need thereof a therapeutically effective amount of a pharmaceutical composition comprising a peptide having anti-inflammatory activity according to the principles of the invention and a pharmaceutically acceptable carrier.

As anti-inflammatory agents, the peptides of the invention are expected to be efficacious in all diseases, disorders, or conditions that involve inflammation or inflammatory activity.

According to some embodiments, the subject to be treated or protected with the pharmaceutical compositions comprising the peptides of the invention is a mammal. According to other embodiments, the mammal is a human. Treatment or protection against inflammation may be accomplished in the fetus, newborn, child, adolescent as well as in adults and old persons, whether the inflammation to be treated is spontaneous, chronic, of traumatic etiology, a congenital defect or a teratogenic phenomenon.

According to some embodiments, the inflammation is associated with an inflammatory disease, disorder or condition.

According to other embodiments, the inflammatory disease is selected from the group consisting of chronic inflammatory disease and acute inflammatory disease.

According to additional embodiments, the inflammation to be treated by the pharmaceutical compositions of the invention is associated with hypersensitivity. According to further embodiments, the hypersensitivity is selected from the group consisting of immediate hypersensitivity, antibody mediated hypersensitivity, immune complex mediated hypersensitivity, T lymphocyte mediated hypersensitivity and delayed type hypersensitivity.

According to further embodiments, the inflammation to be treated by the pharmaceutical compositions of the invention is associated with autoimmune disease. According to other embodiments, the autoimmune disease is selected from the group consisting of cardiovascular disease, rheumatoid disease, glandular disease, gastrointestinal disease, cutaneous disease, hepatic disease, neurological disease, muscular disease, nephritic disease, disease related to reproduction, connective tissue disease and systemic disease.

According to additional embodiments, the inflammation to be treated by the pharmaceutical compositions of the present invention is associated with chronic degenerative neurological disease. The neurological disease include, but not limited to, neurodegenerative disease, multiple sclerosis, Alzheimer's disease, Parkinson's disease, myasthenia gravis, motor neuropathy, Guillain-Barre syndrome, autoimmune neuropathy, Lambert-Eaton myasthenic syndrome, paraneoplastic neurological disease, paraneoplastic cerebellar atrophy, non-paraneoplastic stiff man syndrome, progressive cerebellar atrophy, Rasmussen's encephalitis, amyotrophic lateral sclerosis, Sydeham chorea, Gilles de la Tourette syndrome, autoimmune polyendocrinopathy, dysimmune neuropathy, acquired neuromyotonia, arthrogryposis multiplex, optic neuritis and stiff-man syndrome.

According to some embodiments, the inflammation to be treated by the pharmaceutical compositions of the invention is associated with an infectious disease. According to additional embodiments, the infectious disease is selected from the group consisting of chronic infectious disease, subacute infectious disease, acute infectious disease, viral disease, bacterial disease, protozoan disease, parasitic disease, fungal disease, mycoplasma disease and prion disease.

According to further embodiments, the inflammation to be treated is associated with a disease associated with transplantation of a graft. The disease associated with transplantation of a graft is selected from the group consisting of graft rejection, chronic graft rejection, subacute graft rejection, hyperacute graft rejection, acute graft rejection and graft versus host disease. According to additional embodiments, the graft is selected from the group consisting of a cellular graft, a tissue graft, an organ graft and an appendage graft.

According to other embodiments, the inflammation to be treated is associated with an allergic disease. According to some embodiments, the allergic disease is selected from the group consisting of asthma, hives, urticaria, pollen allergy, dust mite allergy, venom allergy, cosmetics allergy, latex allergy, chemical allergy, drug allergy, insect bite allergy, animal dander allergy, plant allergy and food allergy.

According to further embodiments, the inflammation to be treated by the pharmaceutical compositions of the invention is associated with a tumor. According to additional embodiments, the tumor is selected from the group consisting of a malignant tumor, a benign tumor, a solid tumor, a metastatic tumor and a non-solid tumor.

According to another embodiment, the inflammation is associated with septic shock.

According to further embodiment, the inflammation to be treated is associated with anaphylactic shock.

According to yet further embodiment, the inflammation to be treated is associated with toxic shock syndrome.

According to additional embodiments, the inflammation to be treated is associated with a prosthetic implant. According to some embodiments, the prosthetic implant is selected from the group consisting of a breast implant, a silicone implant, a dental implant, a penile implant, a cardiac implant, an artificial joint, a bone fracture repair device, a bone replacement implant, a drug delivery implant, a catheter, a pacemaker and a respirator tube.

According to other embodiments, the inflammation to be treated is associated with an injury. According to some embodiments, the injury is selected from the group consisting of an abrasion, a bruise, a cut, a puncture wound, a laceration, an impact wound, a concussion, a contusion, a thermal burn, frostbite, a chemical burn, a sunburn, a desiccation, a radiation burn a radioactivity burn, smoke inhalation, a torn muscle, a pulled muscle, a torn tendon, a pulled tendon, a pulled ligament, a torn ligament, a hyperextension, a torn cartilage, a bone fracture, a pinched nerve and a gunshot wound.

According to further embodiments, the inflammation to be treated is a musculo-skeletal inflammation. According to some embodiments, the musculo-skeletal inflammation is selected from the group consisting of arthritis, muscle inflammation, myositis, a tendon inflammation, tendinitis, a ligament inflammation, a cartilage inflammation, a joint inflammation, a synovial inflammation, carpal tunnel syndrome and a bone inflammation.

A pharmaceutical composition of the invention is administered in a manner compatible with the dosage formulation, and in a therapeutically effective amount. The quantity to be administered depends on the subject to be treated, capacity of the subject's blood hemostatic system to utilize the active ingredient, and the degree of inflammation required to be eradicated. Precise amounts of active ingredient required to be administered depend on the judgment of the practitioner and are peculiar to each individual.

The methods of treatment according to the present invention include both therapeutic and prophylactic utility. Thus, the pharmaceutical compositions comprising the peptides of the invention can be administered prior to the occurrence of an inflammation or can be administered after the inflammation has appeared.

According to some embodiments, repeated application may enhance the anti-inflammatory activity of the peptides of the invention and may be required in some applications. Additionally, the peptides of the invention can be administered alone or in conjunction with other therapeutic modalities. Thus, it is appropriate to administer the peptides of the invention as part of a treatment regimen involving other therapies, such as surgery and/or drug therapy.

### EXAMPLE 1

### Isolation and Identification of the Various Peptides in Wound Fluids

Sterile pads were put on diabetic leg ulcers for 1 hour. Thereafter, the pads were removed, placed into sterile tubes containing 10 ml of PBS, and subjected to freezing. After defrosting the fluids, the pads were discarded, the fluids were centrifuged, and the supernatants were passed through a polycarbonate membrane sieve of 5 kDa. Materials having an apparent molecular weight higher than 5 kDa were discarded. Materials having an apparent molecular weight lower than 5 kDa were frozen and subjected to amino acid analysis as described below.

The peptides were resolved by reverse-phase chromatography on 0.1×300-mm fused silica capillaries (J&W, 100 micrometer ID) home-filled with porous R2 (Perspective). The peptides were eluted using an 80-min linear gradient of 5 to 95% acetonitrile with 0.1% acetic acid in water at a flow rate of about 1 μl/min. The liquid from the column was electrosprayed into an ion-trap mass spectrometer (LCQ, Finnigan, San Jose, Calif., USA). Mass spectrometry (MS) was performed in the positive ion mode using repetitively full MS scan followed by collision induces dissociation (CID) of the most dominant ion selected from the first MS scan. The mass spectrometry data was compared to simulated proteolysis and to the CID of the proteins in the “nr” database (NCBI) using the Sequest software (J. Eng and J. Yates, University of Washington and Finnigan, San Jose, Calif., USA).

The identified peptides were then synthesized at Genemed Synthesis, Inc. (South San Francisco, Calif., USA).

TABLE 1

Amino acid sequence of peptides 1-10 and
their molecular origin.

| | | | |
|---|---|---|---|
| Peptide Number | Sequence | Derived from | Residue No. |

| | | | |
|---|---|---|---|
| 1 (SEQ ID NO:1) | MIESSMN | LRP5 | 827-833 |
| 2 (SEQ ID NO:2) | LSALEEYTKKLNTQ | Apo A-I | 254-267 |
| 3 (SEQ ID NO:3) | WDNLEKETEGL | Apo A-I | 96-106 |
| 4 (SEQ ID NO:4) | TDYGKDLMEKV | Apo A-II | 42-52 |
| 5 (SEQ ID NO:5) | NPDESSKPNMI | Fibrinogen γ | 95-105 |
| 6 (SEQ ID NO:6) | ELERPGGNEI | Fibrinogen Aα | 260-269 |
| 7 (SEQ ID NO:7) | ALTDMPQM | Fibrinogen Aα | 250-257 |
| 8 (SEQ ID NO:8) | DHSKNPVGV | ADAM 8 | 320-328 |
| 9 (SEQ ID NO:9) | NDADDST | Cadherin 4 | 302-308 |
| 10 (SEQ ID NO:10) | NQGEESAEI | Calcitonin receptor | 470-478 |

Table 1 lists the various peptides isolated from wound fluids. As indicated in Table 1, the naturally occurring peptides identified in wound fluids originated from the LDL receptor related protein 5 (LRP5), Apo A-I, Apo A-II, fibrinogen γ chain, fibrinogen Aα, ADAM 8, cadherin 4, and calcitonin receptor. It should be noted that peptides 2 and 3 are both derived from Apo A-I, however, while peptide 2 is derived from the carboxyl terminus of the precursor of Apo A-I, namely from the prepro Apo A-I, peptide 3 is derived from the mature Apo A-I. In addition, peptide 5 is derived from the fibrinogen γ chain. This peptide is different from the previously published peptides derived from fibrinogen γ chain as it contains amino acid residues 95-105.

ADAM 8 is a member of cell surface proteins characterized by a disintegrin and a metalloprotease domain (ADAM). The extracellular region of ADAM 8 shows significant amino acid sequence homology to hemorrhagic snake venom proteins. Peptide No. 8 is derived from the metalloprotease region.

Cadherin 4 is a calcium-dependent cell-cell adhesion glycoprotein comprised of five extracellular cadherin repeats, a transmembrane region, and a highly conserved cytoplasmic tail. Based on studies in chicken and mouse, cadherin 4 was implicated to play an important role in brain segmentation and neuronal outgrowth. In addition, a role in kidney and muscle development was indicated. Peptide No. 9 is derived from one of the five extracellular repeats.

Calcitonin is a 32 amino acid peptide hormone that is essential to calcium and phosphorus metabolism. In bone, calcitonin suppresses re-absorption of bone by inhibiting the activity of osteoclasts. In the kidney, however, calcitonin inhibits tubular re-absorption of calcium and phosphorus leading to an increased rate of their loss in the urine. The calcitonin receptor (CTR) is a member of the seven-transmembrane G protein-coupled receptor family. Using transgenic mouse models, the CTR has been shown to be expressed in the limb buds, cornea, retina, muscle, and in the nervous system during development of the fetus, suggesting a key role for the calcitonin system in morphogenesis.

### EXAMPLE 2

### The Anti-Inflammatory Activity of the Peptides

(a) Delayed type hypersensitivity assay

Groups of 5 female inbred BALB/c mice (The Jackson Laboratory, Bar Harbor, Me.) were sensitized on the shaved abdominal skin with 2% oxazolone (100 μl) dissolved in acetone/olive oil [4:1 (vol/vol)] applied topically. Delayed type hypersensitivity (DTH) was elicited 5 days later by challenging the mice with 0.5% oxazolone in acetone/olive oil (10 μl given topically to each side of the ear). A constant area of the ear was measured immediately before and 24 hours after the challenge using a Mitutoyo engineer's micrometer. The percent inhibition of DTH is calculated as follows:

% Inhibition=[1-{(treated−negative control)/(positive control−negative control)}]×100.

The positive control is the DTH reaction to oxazolone elicited in sensitized mice without treatment. The negative control is the background swelling produced by the oxazolone antigen in naïve non-sensitized mice.

Treatments with Peptides:

Peptides Nos. 1-5 were given to the mice by mini perfusion pumps (alzet osmotic pump model 2001), which contained the indicated peptide (40 μg; 200 μl). The pumps were implanted under the mouse skin one day before oxazolone sensitization and they released the peptide in a rate of 1 μl per hour (0.2 μg/hour) for 7 days.

Peptides Nos. 6-10 were given to the mice by subcutaneously injection (1 μg or 10 μg per mouse), every day, starting at one day before oxazolone sensitization and ending at day 5 post DTH initiation.

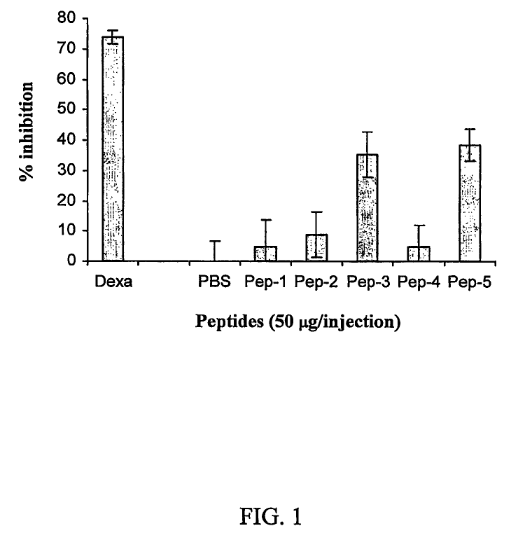

FIG. 1 shows the effect of peptides 1-5 on DTH response. As shown in FIG. 1, peptides Nos. 1, 2, and 4, derived from the LDL receptor, Apo A-I (amino acid residues 254-267), and from Apo A-II, respectively, hardly inhibited DTH response. However, peptides Nos. 3 and 5, which are derived from Apo A-I (amino acid residues 96-106) and from fibrinogen γ chain, respectively, significantly inhibited (˜40%) DTH response. Dexametazone was used as a positive control and inhibited the DTH response by ˜70%.

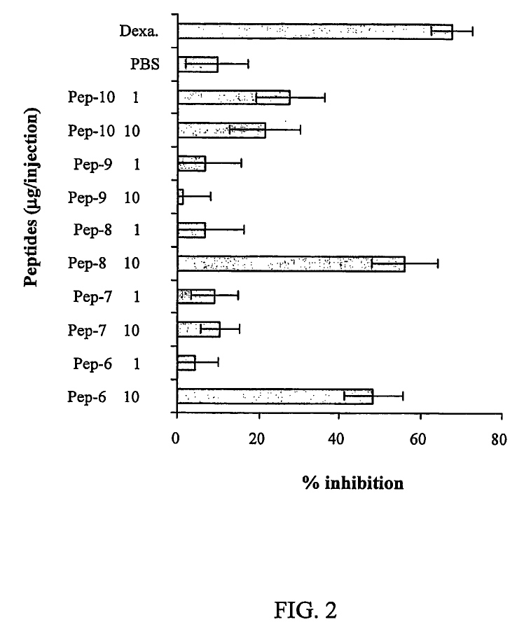

FIG. 2 shows the effect of peptides 6-10 on DTH response. As shown in FIG. 2, peptides 7 and 9, derived from the Fibrinogen Aα chain (amino acid residues 250-257), and from Cadherin 4, respectively, hardly inhibited DTH response. However, peptides Nos. 6, 8, and 10, which are derived from Fibrinogen Aα chain (amino acid residues 260-269), ADAM8, and from Calcitonin receptor inhibited the DTH response by 50%, 60%, and 30%, respectively. Dexametazone was also used as a positive control and inhibited the DTH response by ˜70%.

### (b) Adoptive Transfer DTH Assay

Female inbred BALB/c mice (The Jackson Laboratory, Bar Harbor, Me.) were sensitized on the shaved abdominal skin with 2% oxazalone (100 μl) dissolved in acetone/olive oil [4:1 (vol/vol)]. Two donor mice were sensitized for one recipient mouse. On day 5, lymph nodes were removed and single-cell suspensions of lymph node cells were prepared. Lymph node cells were incubated (20 hours) with either peptide No. 3 or peptide No. 5 (10 rig/ml) in RPMI 1640 containing 10% FCS. After the incubation, the cells were washed, resuspended with PBS and injected intravenously (40 million cells per mouse) to syngeneic recipient mice. The mice were immediately challenged with 0.5% oxazalone in acetone/olive oil (10 μl given topically to each side of the ear) to elicit DTH reactions. The increment of ear swelling was measured after 24 hours.

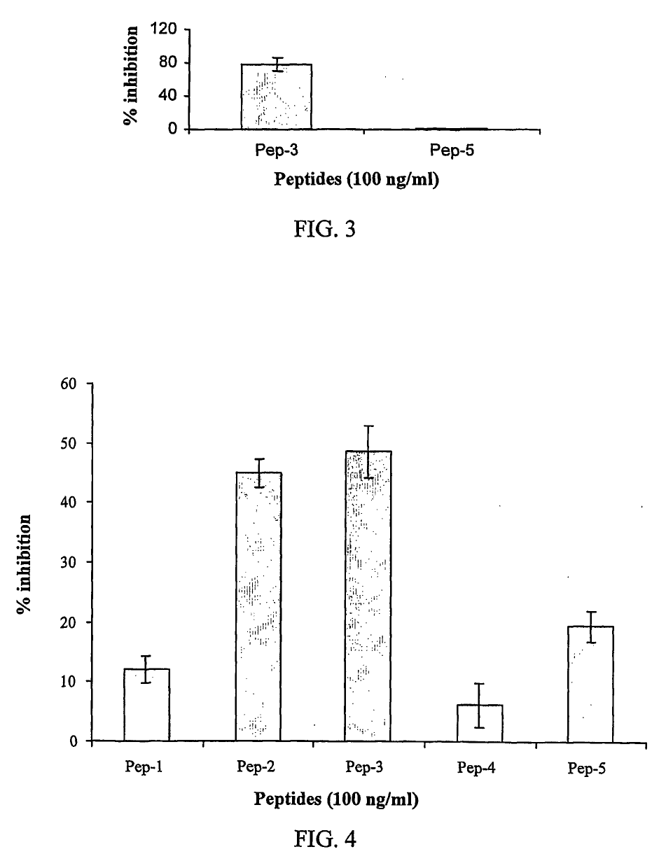

FIG. 3 shows the effect of peptides Nos. 3 and 5 on the adoptive transfer DTH response. As shown in FIG. 3, treatment of lymph node cells with peptide No. 3 was found to inhibit the adoptive transfer DTH response by 80%. Peptide No. 5 did not exhibit a significant inhibition under these experimental conditions.

### (c) T Cell Adhesion to Immobilized Fibronectin

Peripheral blood lymphocytes (PBL) were isolated on Ficoll gradient, washed, resuspended in PBS containing 3% heat-inactivated FCS, and incubated (45 min, 37° C., 7% CO₂-humidified atmosphere) on nylon-wool columns (NovaMed; Jerusalem, Israel). Non-adherent cells were eluted, washed, and platelets were removed by centrifugation (700 rpm, 15 min, 18° C.). Residual monocytes were removed by incubating (2 hr, 37° C.) the cells on tissue culture plates, and the non-adherent monocytes were collected. The PBL thus obtained contained >95% CD3+ cells.

Adhesion of T cells to fibronectin (FN) was analysed as follow: flat-bottomed microtiter (96-well) plates were pre-coated with FN (0.5 μg/well), and then blocked with 0.1% BSA in phosphate-buffered saline (PBS). ⁵¹-[Cr]-labeled PBL were either left untreated or were pre-incubated (30 min, 37° C.) with the indicated peptides (100 ng/ml) before exposure to phorbol 12-myristate 13-acetate (PMA) (50 ng/ml for usually 30 min at 37° C.). The cells (10⁵ cells in 100 μl RPMI containing 0.1% BSA) were then added to the FN-coated plates, incubated (30 min, 37° C.), and then washed. Adherent cells were lysed, and the resulting supernatants were collected and counted in a γ-counter. The results are expressed as the mean percentage (±SD) of adhered T cells out of the total added cells from quadruplicate wells for each experimental group.

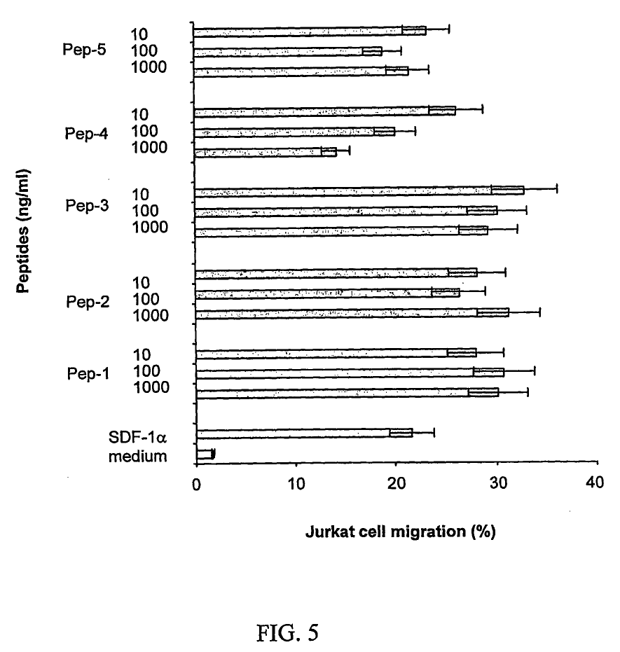

FIG. 4 shows the effect of peptides No. 1-5 on the PMA-induced T cell adhesion to FN. As shown in FIG. 4, peptides Nos. 2 and 3 exerted significant inhibition (45-50%) on the PMA-induced T cell adhesion to FN. Peptide No. 5 was able to inhibit the T cell adhesion by 20%, while peptides Nos. 1 and 4 showed only slight inhibition (up to 10%).

### (d) Chemotaxis Assay

The migration of ⁵¹-[Cr]-labeled T cells was examined in 48-well Transwell chemotaxis apparatus (6.5-mm diameter; Corning, N.Y.), consisting of two compartments separated by polycarbonate filters (5 μm pore size) pretreated (1 hr, 37° C.) with FN (25 μg/ml). ⁵¹-[Cr]-labeled T cells (2×10⁵ in 100 μl of RPMI containing 0.1% BSA and antibiotics), which were pre-incubated with the indicated concentrations of peptides (30 min, 37° C.), were added to the upper chambers. The bottom chambers contained 0.6 ml of the same media, with or without human Stromal cell-Derived Factor-1α (SDF-1α; 250 ng/ml). After incubation at 37° C. for 3 hr (7.5% CO₂-humidified atmosphere), cells that had transmigrated into the lower wells were collected, centrifuged, and lysed (in 100 μl of distilled water containing 1 M NaOH and 0.1% Triton X-100), and the radioactivity in the resulting supernatants was determined with a γ-counter. The percentage (±SD) of cell migration was calculated as the radioactivity counts in the lysates of the lower chambers (representative of the migrating cells) divided by the total counts (representative of 2×10⁵ cells).

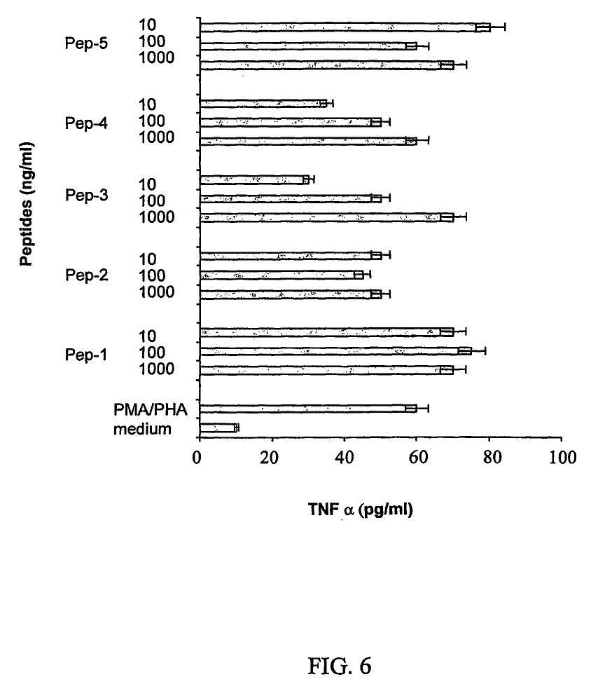

FIG. 5 shows the effect of the peptides Nos. 1-5 on the T cell transmigration toward SDF-1α. Peptides Nos. 1, 2, 3, and 5 did not show a significant inhibition on the T cell transmigration at the concentration range (10-1000 ng/ml) used. However, peptide No. 4 at a concentration of 1000 ng/ml was found to inhibit T cell migration by 30%.

### (e) TNFα Secretion by PMA/PHA Induced Jurkat and THP-1 Cells

Jurkat cells human T cell lymphoma line) (2×10⁶ cells/ml) cultured in RPMI-1640 supplemented with 10% FCS were activated with PMA (50 ng/ml) and phytohaemagglutinin PHA (4 □g/ml) for 6 hours at 37° C. (7.5% CO₂-humidified atmosphere). Thereafter, the cells were washed, plated on 24-well plates (10⁶ cells/well in 0.5 ml), and incubated (1 hour at 37° C., 7.5% CO₂-humidified atmosphere) with the indicated concentrations of peptides 1-5. Following the incubation, THP-1 cells (human monocytic cell line derived from a patient with acute monocytic leukaemia; 1.25×10⁵ cells/0.5 ml/well) were added to the wells. After 48 hours, the supernatants were collected and their TNF□ content was determined by ELISA using anti TNF□ mAb (Pharmingen; San Diego, Calif.) according to the manufacturer's instructions.

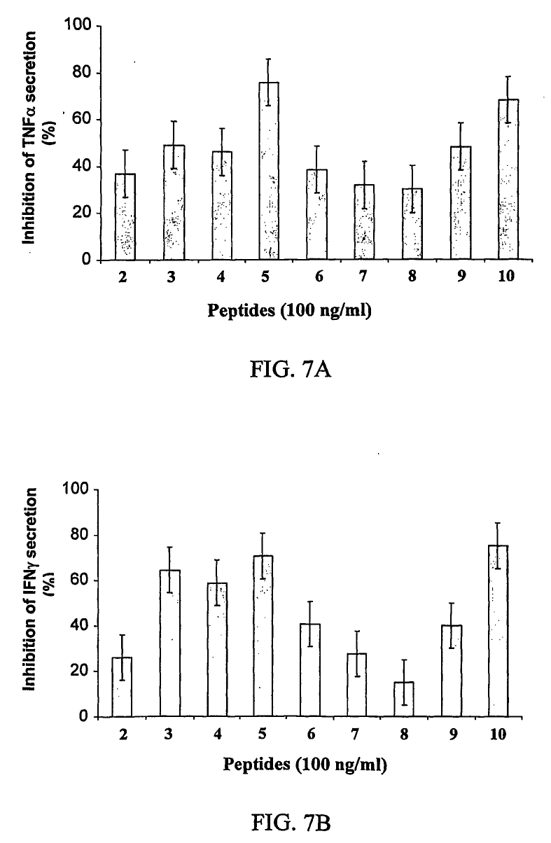

FIG. 6 shows the effect of peptides Nos. 1-5 on TNFα secretion by PMA/PHA induced Jurkat and THP-1 cells. As shown in FIG. 6, treatment of Jurkat cells with PMA/PHA induced monocytes to secrete TNFα at a level of ˜60 ng/ml. Peptides Nos. 1 and 5 at a concentration range of 1-1000 ng/ml did not exert a significant inhibition on TNFα secretion by Jurkat and THP-1 cells. However, peptides Nos. 2, 3, and 4 (at a concentration of 1 μg/ml) were shown to inhibit TNFα secretion by 10, 50, and 40%, respectively, compared to PMA/PHA alone.

### (f) TNFα and IFNγ Secretion by Anti CD3 Activated T Cells

Human T cells were purified and maintained in culture (RPMI containing 10% FCS, 1% Pyruvate, 1% glutamine, 1% antibiotics, 7.5% CO₂, humidified atmosphere) for 15 hr before they were transferred to RPMI and activated (2 hr, 37° C.) with the indicated concentrations of the peptides. The cells were then plated (1.5×10⁶ cells/0.5 ml/well) in 24 wells plates (non-tissue culture grade) precoated with anti-CD3 mAb in order to stimulate the cells to secrete cytokines. After 24 hours, the supernatants were collected and their cytokine content (TNFα and IFNγ) was determined by ELISA using anti-IFNγ and anti TNFα mAb (Pharmingen; San Diego, Calif.) according to the manufacturer's instructions.

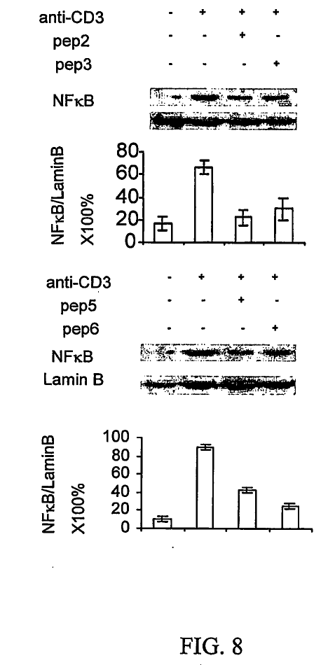

FIG. 7A shows the effect of peptides Nos. 2-10, at a concentration of 100 ng/ml, on anti-CD3 antibody induced TNFα secretion from T cells. As shown in FIG. 7A, peptide Nos. 2, 3, 4, 5, 6, 7, 8, 9, and 10 were found to inhibit anti-CD3 antibody induced TNFα secretion from T cells by 40%, 50%, ˜50%, 85%, 40%, 30%, 30%, ˜50%, and by ˜70%, respectively.

FIG. 7B shows the effect of peptides Nos. 2-10 on anti-CD3 antibody induced INFγ secretion from T cells. As shown in FIG. 7B, peptides Nos. 2, 3, 4, 5, 6, 7, 8, 9, and 10 were found to inhibit anti-CD3 antibody induced IFNγ secretion from T cells by ˜30%, 65%, ˜50%, 60%, 40%, 30%, 15%, 40%, and by 75%.

### (g) Nuclear Translocation of NF-κB in T Cells

Activation and nuclear translocation of NF-κB is an essential step in the regulation of gene expression and secretion of various pro-inflammatory cytokines in leukocytes, including T cells (Ghosh, S., et al. 1998. Annu. Rev. Immunol. 16, 225-260).

To address the modulatory role of the wound fluid peptides on the signal transduction cascade leading to regulation of T-cell secretion of TNF-α(and IFN-γ, the nuclear translocation of NF-κB was detected by probing nuclear and cytoplasmic T-cell extracts using mAb specific for the p65 subunit of NF-κB. The nuclear protein Lamin B was used as continuatively expressed control protein for the quantification of protein amounts.

### Material and Methods:

Human T cells. T cells were purified from the peripheral blood of healthy human donors (Blood Bank; Tel-Hashomer, Israel). The whole blood was incubated (20 min, 22° C.) with RosetteSep™ human T-cell enrichment cocktail (StemCell Technologies, Vancouver, BC, Canada). The remaining unsedimented cells were then loaded onto Lymphocyte Separation Medium (ICN Biomedicals; Belgium), isolated by density centrifugation, and washed with PBS. The purified cells (>95% CD3⁺ T cells) so obtained were cultured in RPMI containing antibiotics and 10% heat-inactivated FCS. Western blot analysis of T-cell nuclear extracts. Purified T cells (5×10⁶) were preincubated with the peptides (100 ng/ml) for 2 hours (37° C. in a 7% CO₂, humidified atmosphere). The cells were then replated in 24-well plates pre-coated with anti-CD3 mAb for 24 hr (37° C. in a 7% CO₂, humidified atmosphere). T cells were lysed in 10 mM HEPES, 1.5 mM MgCl₂, 1 mM dithiothreitol (DTT), 1 mM PMSF, 0.5% Nonidet P-40. The lysates were incubated on ice for 10 min and centrifuged at 2000 rpm for 10 min at 4° C. The supernatants (cytoplasmic extracts) were transferred and the pellet (nuclei) was suspended in buffer containing 30 mM HEPES, 450 mM NaCl, 25% Glycerol, 0.5 mM EDTA, 6 mM DTT, 12 mM MgCl₂ 1 mM PMSF, 10 μg/ml leupeptin, 10 μg/ml pepstatin, 1% phosphatase inhibitor cocktail, and the suspension was incubated on ice for 30 min. The lysates were cleared by centrifugation (30 min, 14×10³ rpm, 4° C.), and the resulting supernatants analyzed for protein content. Sample buffer was then added and, after boiling, the samples, containing equal amounts of proteins, were separated on 10% SDS-PAGE and transferred to nitrocellulose membranes. The membranes were blocked with TBST buffer containing low-fat milk (5%), Tris pH 7.5 (20 mM), NaCl (135 mM) and Tween 20 (0.1%)], and probed with either anti-NF-κB (diluted 1:1000) or anti-Lamin B (diluted 1:1000) in the same buffer. Immunoreactive protein bands were visualized using labeled secondary antibodies and the enhanced ECL system.

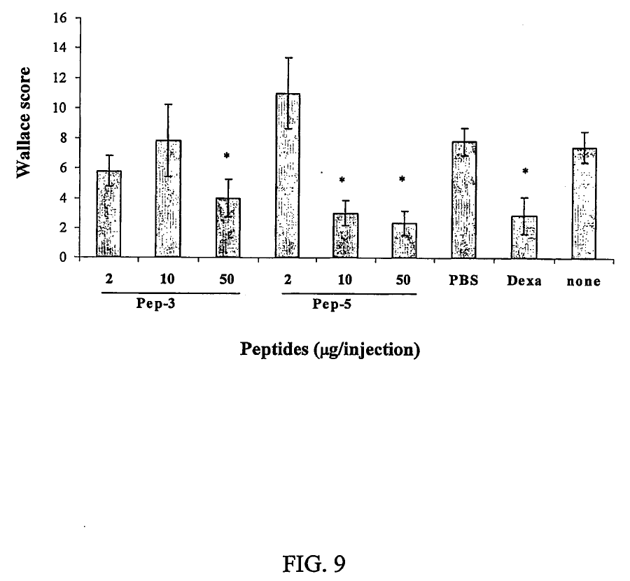

As shown in FIG. 8, pre-treatment of T cells with peptides No. 2, 3, 5 and 6, followed by their exposure to anti-CD3, caused a significant down-regulation of NF-κB translocation to the nucleus. The reduction in NF-κB levels in activated T cells in response to the peptides support the possibility that the NF-κB pathway is a mechanism through which the peptides can reduce T-cell activation by down-regulating the secretion of IFNγ and TNFα.

### (h) Inflammatory Bowel Diseases (IBD)

In this study the effect of wound fluid peptides on colitis in an experimental animal model was studied.

BALB/C mice, which were anesthetized for 5-10 min, received by intrarectal administration, 30 μl of a solution of 2,4,6-trinitrobenzene sulfonic acid (TNBS; 100 mg/kg) dissolved in 0.9% NaCl and mixed with an equal volume of ethanol (50% ethanol). Animals were killed 7 days after TNBS administration.

Wound fluid peptides were administered once daily by subcutaneous (SC) administration starting 1 day before the induction of colitis and every day for the next 7 days as follows:

| Compound | Dose/mouse | | Administration | Frequency |
|---|---|---|---|---|
| Pep-3 | 2 | μg | SC | Daily |
| Pep-3 | 10 | μg | SC | Daily |
| Pep-3 | 50 | μg | SC | Daily |
| Pep-5 | 2 | μg | SC | Daily |
| Pep-5 | 10 | μg | SC | Daily |
| Pep-5 | 50 | μg | SC | Daily |
| PBS | | | SC | Daily |
| Dexamethasone | 200 | μg | SC | Daily |
| untreated | | | | |

Treatment with the wound fluid peptides was evaluated at day 7 by determination of macroscopic inflammation scores. The colon of each mouse was examined under a dissecting microscope to evaluate the macroscopic lesions according to the Wallace criteria. The Wallace score lists macroscopic colon lesions on a scale from 0 to 10 based on features reflecting inflammation, such as hyperemia, thickening of the bowel, and extent of ulceration (Wallace, J. L., et al. 1989, Gastroenterology, 96:29).

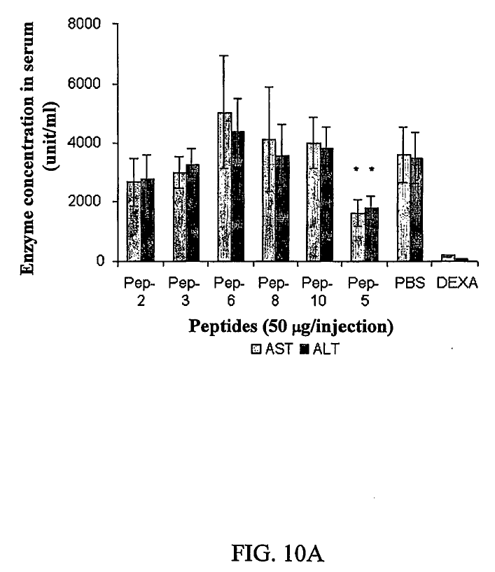

FIG. 9 shows the effect of peptide No. 3 and peptide No. 5 on inflammatory bowel diseases in mice. As shown in FIG. 9, untreated mice showed mild IBD (Wallace score 7.4±0.8). Mice treated SC with peptide No. 3 (50 μg) showed significant resistance to IBD (score of 4.0±1.3; p=0.03). However, mice treated SC with peptide No. 5 showed higher resistance to IBD (10 μg, score of 3.0±0.9, p=0.005; 50 μg, score of 2.3+0.8; p=0.002) compared to those treated with peptide No. 3. The resistance to IBD obtained by peptide No. 5 was comparable to that obtained by SC administration of Dexamethasone (score 2.9±1; p=0.005). These results indicate that the wound fluid peptides No. 3 and No. 5 exert protective and therapeutic effect against an inflammatory disease.

### (i) Con A-Induced Hepatic Injury

Con A induced hepatitis is an experimental animal model of acute hepatitis. Con A directly activates immune cells that secrete inflammatory mediators such as cytokines, chemokines, and acute phase proteins.

Con A (13 mg/kg) was administered i.v. to BALB/c mice 18 hours before analysis (Bonder, C. S. et al. 2004. J. Immunol. 172: 45-53). Mice were intraperitoneally injected with the peptides of the invention 48 and 2 hours before ConA injection.

Blood analysis: 18 hours after Con A injection, blood was obtained by cardiac puncture for detection of serum alanine aminotransferase (ALT) and aspartate aminotransferase (AST). The presence of these two enzymes in serum is used as an index of hepatocellular injury. The detection of ALT and AST was performed by a commercial kit for ALT and AST (Bayer Diagnostics, Tarry town, NY, USA) using an automated Monarch Monoanalyzer 2000 (Allied, Lexington, Mass.).

| | | Dose/mouse; 2 injections: | |
|---|---|---|---|
| Group* | Material | 48 and 2 hours before ConA | |
| 1 | Pep-2 | 50 | μg |
| 2 | Pep-3 | 50 | μg |
| 3 | Pep-6 | 50 | μg |
| 5 | Pep-8 | 50 | μg |
| 6 | Pep-5 | 50 | μg |
| 7 | PBS | 50 | μg |
| 8 | Dexamethasone | | |
| | 200 μg | | |

*Each group consists of 6 mice.

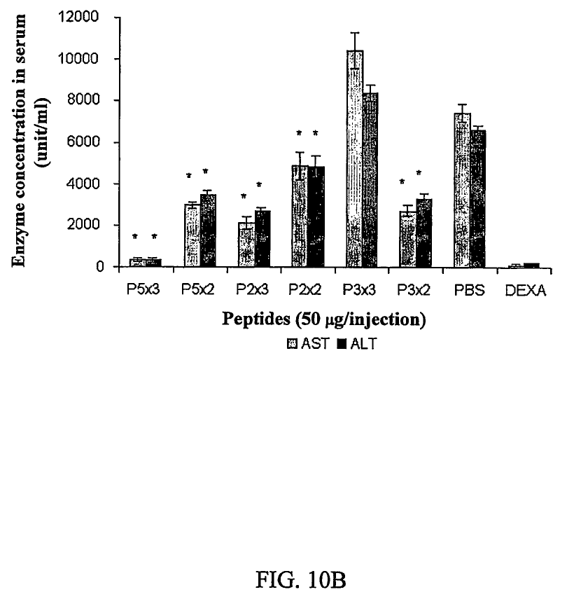

FIG. 10A shows the effect of peptides No. 2, 3, 5, 6, 8, and 10 on Con A induced hepatitis in mice. As shown in FIG. 10A, the concentration of ALT and AST in serum of mice treated with peptide No. 5 was significantly lower (˜50%) than that of untreated mice.

### Exp. 2:

| | | Dose/mouse; | Dose/mouse; |
|---|---|---|---|
| | | 2 injections: | 3 injections: |
| | | 48 and 2 hours | 72, 48 and 2 hours |
| Group* | Material | before ConA | before [symbol] |
| 1 | Pep-5 | | 50 μg |
| 2 | Pep-5 | 50 μg | |
| 3 | Pep-2 | | 50 μg |
| 4 | Pep-2 | 50 μg | |
| 5 | Pep-3 | | 50 μg |
| 6 | Pep-3 | 50 μg | |
| 7 | PBS | | |
| 8 | Dexamethasone | | |
| | 200 μg | | |

*Each group consists of 6 mice. [symbol]
indicates data missing or illegible when filed

As shown in FIG. 10B, untreated mice showed mild ConA induced hepatitis. Mice treated intraperitoneally with peptide No. 5 or peptide No. 2 showed marked resistance to ConA induced hepatitis, especially after 3 treatments (p0.00001). Mice treated intraperitoneally with peptide hepatitis, especially after 2 treatments (p=0.0001).

### (j) Autoimmune Encephalomyelitis (EAE)

EAE, an experimental model of sclerosis, is induced in rats by intra-peritoneal injection of 2 million T cells activated by myelin basic protein (MBP) peptide (Achiron, A. et al. (2000) J. Autoimmunity 15: 323-330). Clinical EAE is observed 4-5 days after T cell injection, it peaks at days 12-15, and then it gradually subsides. Rats (10 rats/group) are subcutaneously injected with a peptide of the invention (2 doses: 10 μg and 50 μg; 0.1 ml/rat). Injections of the peptides are performed every day, every other day, or once a week. The experiment is repeated twice. After 30 days from the induction of the disease, the rats are sacrificed with CO₂.

The animals are not euthanized upon appearance of the first signs of paralysis as the effect of the peptides on the disease's duration and on its rate of decline are monitored. To assess the severity of EAE, scoring of clinical signs is as follows: O=no signs; 1=loss of tail tonicity; 2=paralysis of hind limbs; 3=paralysis of all four limbs; 4=quadriplegic animal in moribound state; and 5=death caused by EAE.

(k) Adjuvant arthritis (AA)

AA, an experimental model of rheumatoid arthritis, is induced in rats by injecting intradermally at the base of the tail complete Freud's adjuvant (0.1 ml) containing 10 mg/ml Mycobacterium tuberculosis (H37RA; Difco, Detroit, Mich.) under anesthesia (Pentotal) (Cahalon, L., (1997) International Immunology 9: 1517-1522). The onset of AA occurs at day 11, it peaks at day 23-26, and then it gradually subsides. Rats (10 rats/group) are subcutaneously injected with a peptide of the invention (2 doses: 10 μg and 50 μg; 0.1 ml/rat). Injections of the peptides are performed every day, every other day, or once a week. The experiment is repeated twice. After 60 days from the induction of the disease, the rats are sacrificed with CO₂. To assess the severity of arthritis, each paw is scored clinically on a scale of 0-4, based on erythema, swelling and deformity of the joint. The total arthritis score (0-16) is obtained by summarizing the score of all the four joints. Monitoring the clinical signs lasts 2 months after the induction of the disease.

### (l) Stability of Peptide No. 5 to Serum Proteases

The use of peptides as therapeutic drugs has largely been limited by their short half-life in vivo. Because peptides are mainly cleaved by proteases and peptidases, peptide's resistance against these enzymes is of high importance in determining the bioavailability of a peptide.

To test the stability of peptide No. 5 to blood proteases, 100 μl of a 1 mg/ml solution of the peptide were incubated at 37° C. with 40 μl of mouse serum. Samples withdrawn after 0, 2, 4 or 24 hours were precipitated with 200 μl of methanol and centrifuged for 1 min at 10,000×g. The crude solution was analyzed by HPLC equipped with a 220 nm detector. The crude solution was diluted in 0.1% trifluoroacetic acid (TFA), applied on a RP-18^{e} column (Chromolith) (MERCK, Darmstadt, Germany), and the HPLC conditions were as follows: mobile phase A, TFA (0.1%) in water; mobile phase B: TFA (0.1%) in acetonitrile (75%). The flow rate was 3 ml/min and the gradient was 0 to 100% B for the first 10 min, 100% B for additional 5 min, and for the last 5 min the gradient was 100% B to 0.

TABLE 1
Stability of peptide No. 5 to serum proteases.

| | | | | |
|---|---|---|---|---|
| Incubation time | RT | Area | % Area | Height |

Test 1

| | | | | |
|---|---|---|---|---|
| 24 hours | 8.638 | 45802804 | 68.69 | 1966924 |
| 4 hours | 8.676 | 36963990 | 68.94 | 1978949 |
| 2 hours | 8.578 | 44317519 | 75.08 | 2028844 |
| 0 hours | 8.52 | 44000201 | 74.23 | 1931032 |

Test 2

| | | | | |
|---|---|---|---|---|
| 2 hours | 8.41 | 12288671 | 87.32 | 1021387 |
| 0 hours | 8.415 | 16516560 | 90.62 | 1200030 |

As shown in Table 1, approximately 3.5% of the peptide were degraded after 2 hours (test 2) and 7.5% of the peptide were degraded after 4 or 24 hours (test 1) in the presence of mouse serum.

These results indicate that peptide No. 5 is highly resistant to serum proteases.

It will be appreciated by persons skilled in the art that the present invention is not limited by what has been particularly shown and described herein above. Rather the scope of the invention is defined by the claims that follow.

1. A peptide having anti-inflammatory activity comprising a proteolytic fragment of a naturally occurring protein, the peptide present in wound fluids at a site of tissue injury or trauma.

2. The peptide according to claim 1 comprising an amino acid sequence as set forth in any one of SEQ ID NO: 1 to SEQ ID NO:10, an analog, derivative, fragment, conjugate, or a salt thereof.

3. The peptide according to claim 2 comprising the sequence of amino acid residues 827 to 833 of LDL receptor related protein 5 (LRP5) as set forth in SEQ ID NO:1, an analog, derivative, fragment, conjugate, or a salt thereof.

4. The peptide according to claim 2 comprising the sequence of amino acid residues 254 to 267 of apolipoprotein (Apo) A-I as set forth in SEQ ID NO:2, an analog, derivative, fragment, conjugate, or a salt thereof.

5. The peptide according to claim 2 comprising the sequence of amino acid residues 96 to 106 of apolipoprotein (Apo) A-I as set forth in SEQ ID NO:3, an analog, derivative, fragment, conjugate, or a salt thereof.

6. The peptide according to claim 2 comprising the sequence of amino acid residues 42 to 52 of apolipoprotein (Apo) A-II as set forth in SEQ ID NO:4, an analog, derivative, fragment, conjugate, or a salt thereof.

7. The peptide according to claim 2 comprising the sequence of amino acid residues 95 to 105 of fibrinogen γ as set forth in SEQ ID NO:5, an analog, derivative, fragment, conjugate, or a salt thereof.

8. The peptide according to claim 2 comprising the sequence of amino acid residues 260 to 269 of fibrinogen Aα as set forth in SEQ ID NO:6, an analog, derivative, fragment, conjugate, or a salt thereof.

9. The peptide according to claim 2 comprising the sequence of amino acid residues 250 to 257 of fibrinogen Aα as set forth in SEQ ID NO:7, an analog, derivative, fragment, conjugate, or a salt thereof.

10. The peptide according to claim 2 comprising the sequence of amino acid residues 320 to 328 of a disintegrin and metalloprotease protein (ADAM) 8 as set forth in SEQ ID NO:8, an analog, derivative, fragment, conjugate, or a salt thereof.

11. The peptide according to claim 2 comprising the sequence of amino acid residues 302 to 308 of cadherin 4 as set forth in SEQ ID NO:9, an analog, derivative, fragment, conjugate, or a salt thereof.

12. The peptide according to claim 2 comprising the sequence of amino acid residues 470 to 478 of calcitonin receptor as set forth in SEQ ID NO:10, an analog, derivative, fragment, conjugate, or a salt thereof.

13. A pharmaceutical composition comprising as an active ingredient a peptide having anti-inflammatory activity comprising a proteolytic fragment of a naturally occurring protein, the peptide present in wound fluids at a site of tissue injury or trauma and a pharmaceutically acceptable carrier.

14. The pharmaceutical composition according to claim 13, wherein the peptide comprises an amino acid sequence as set forth in any one of SEQ ID NO: 1 to SEQ ID NO:10, an analog, derivative, fragment, conjugate, or a salt thereof.

15. The pharmaceutical composition according to claim 13, wherein the composition is formulated in a form selected from the group consisting of pellets, tablets, capsules, solutions, suspensions, emulsions, powders, gels, creams, suppositories, and depots.

16. A method for treating or protecting against an inflammation in a subject comprising administering to the subject in need thereof a therapeutically effective amount of a pharmaceutical composition comprising as an active ingredient a peptide having anti-inflammatory activity comprising a proteolytic fragment of a naturally occurring protein, the peptide present in wound fluids at a site of tissue injury or trauma, and a pharmaceutically acceptable carrier.

17. The method according to claim 16, wherein the peptide comprises an amino acid sequence as set forth in any one of SEQ ID NO: 1 to SEQ ID NO:10, an analog, derivative, fragment, conjugate, or a salt thereof.

18. The method according to claim 16, wherein the subject is a mammal.

19. The method according to claim 18, wherein the mammal is a human.

20. The method according to claim 16, wherein the subject is a non-mammalian vertebrate.

21. The method according to claim 16, wherein the route of administering the pharmaceutical composition is selected from the group consisting of parenteral, oral, rectal, vaginal, topical, pulmonary, intranasal, buccal, intradermal, ophthalmic intravenous, intraarterial, subcutaneous, intraperitoneal, intramuscular, intraventricular, and intralesional administration.

22. The method according to claim 16, wherein the inflammation is associated with an inflammatory disease, disorder or condition.

23. The method according to claim 22, wherein the inflammatory disease is selected from the group consisting of chronic inflammatory disease and acute inflammatory disease.

24. The method according to claim 16, wherein the inflammation is associated with hypersensitivity.

25. The method according to claim 24, wherein the hypersensitivity is selected from the group consisting of immediate hypersensitivity, antibody mediated hypersensitivity, immune complex mediated hypersensitivity, T lymphocyte mediated hypersensitivity and delayed type hypersensitivity.

26. The method according to claim 16, wherein the inflammation is associated with autoimmune disease.

27. The method according to claim 26, wherein the autoimmune disease is selected from the group consisting of cardiovascular disease, rheumatoid disease, glandular disease, gastrointestinal disease, cutaneous disease, hepatic disease, neurological disease, muscular disease, nephritic disease, disease related to reproduction, connective tissue disease and systemic disease.

28. The method according to claim 16, wherein the inflammation is associated with chronic degenerative neurological disease.

29. The method according to claim 28, wherein the neurological disease is selected from the group consisting of neurodegenerative disease, multiple sclerosis, Alzheimer's disease, Parkinson's disease, myasthenia gravis, motor neuropathy, Guillain-Barre syndrome, autoimmune neuropathy, Lambert-Eaton myasthenic syndrome, paraneoplastic neurological disease, paraneoplastic cerebellar atrophy, non-paraneoplastic stiff man syndrome, progressive cerebellar atrophy, Rasmussen's encephalitis, amyotrophic lateral sclerosis, Sydeham chorea, Tourette syndrome, autoimmune polyendocrinopathy, dysimmune neuropathy, acquired neuromyotonia, arthrogryposis multiplex, optic neuritis and stiff-man syndrome.

30. The method according to claim 16, wherein the inflammation is associated with an infectious disease.

31. The method according to claim 30, wherein the infectious disease is selected from the group consisting of chronic infectious disease, subacute infectious disease, acute infectious disease, viral disease, bacterial disease, protozoan disease, parasitic disease, fungal disease, mycoplasma disease and prion disease.

32. The method according to claim 16, wherein the inflammation is associated with a disease associated with transplantation of a graft.

33. The method according to claim 32, wherein the disease is selected from the group consisting of graft rejection, chronic graft rejection, subacute graft rejection, hyperacute graft rejection, acute graft rejection and graft versus host disease.

34. The method according to claim 32, wherein the graft is selected from the group consisting of a cellular graft, a tissue graft, an organ graft and an appendage graft.

35. The method according to claim 16, wherein the inflammation is associated with an allergic disease.

36. The method according to claim 35, wherein the allergic disease is selected from the group consisting of asthma, hives, urticaria, pollen allergy, dust mite allergy, venom allergy, cosmetics allergy, latex allergy, chemical allergy, drug allergy, insect bite allergy, animal dander allergy, plant allergy and food allergy.

37. The method according to claim 16, wherein the inflammation is associated with a tumor.

38. The method according to claim 37, wherein the tumor is selected from the group consisting of a malignant tumor, a benign tumor, a solid tumor, a metastatic tumor and a non-solid tumor.

39. The method according to claim 16, wherein the inflammation is associated with septic shock.

40. The method according to claim 16, wherein the inflammation is associated with anaphylactic shock.

41. The method according to claim 16, wherein the inflammation is associated with toxic shock syndrome.

42. The method according to claim 16, wherein the inflammation is associated with a prosthetic implant.

43. The method according to claim 42, wherein said prosthetic implant is selected from the group consisting of a breast implant, a silicone implant, a dental implant, a penile implant, a cardiac implant, an artificial joint, a bone fracture repair device, a bone replacement implant, a drug delivery implant, a catheter, a pacemaker and a respirator tube.

44. The method according to claim 16, wherein the inflammation is associated with an injury.

45. The method according to claim 44, wherein the injury is selected from the group consisting of an abrasion, a bruise, a cut, a puncture wound, a laceration, an impact wound, a concussion, a contusion, a thermal burn, frostbite, a chemical burn, a sunburn, a desiccation, a radiation burn, a radioactivity burn, smoke inhalation, a torn muscle, a pulled muscle, a torn tendon, a pulled tendon, a pulled ligament, a torn ligament, a hyperextension, a torn cartilage, a bone fracture, a pinched nerve and a gunshot wound.

46. The method according to claim 45, wherein the inflammation is a musculo-skeletal inflammation.

47. The method according to claim 46, wherein the musculo-skeletal inflammation is selected from the group consisting of arthritis, muscle inflammation, myositis, a tendon inflammation, tendinitis, a ligament inflammation, a cartilage inflammation, a joint inflammation, a synovial inflammation, carpal tunnel syndrome and a bone inflammation.


SEQUENCE LISTING (10 sequences)

SEQ ID NO: 1 - protein, 7 aa, Artificial sequence
  Synthetic peptide
MIESSNM

SEQ ID NO: 2 - protein, 14 aa, Artificial sequence
  Synthetic peptide
LSALEEYTKKLNTQ

SEQ ID NO: 3 - protein, 11 aa, Artificial sequence
  Synthetic peptide
WDNLEKETEGL

SEQ ID NO: 4 - protein, 11 aa, Artificial sequence
  Synthetic peptide
TDYGKDLMEKV

SEQ ID NO: 5 - protein, 11 aa, Artificial sequence
  Synthetic peptide
NPDESSKPNMI

SEQ ID NO: 6 - protein, 10 aa, Artificial sequence
  Synthetic peptide
ELERPGGNEI

SEQ ID NO: 7 - protein, 8 aa, Artificial sequence
  Synthetic peptide
ALTDMPQM

SEQ ID NO: 8 - protein, 9 aa, Artificial sequence
  Synthetic peptide
DHSKNPVGV

SEQ ID NO: 9 - protein, 7 aa, Artificial sequence
  Synthetic peptide
NDADDST

SEQ ID NO: 10 - protein, 9 aa, Artificial sequence
  Synthetic peptide
NQGEESAEI